(19) United States
(12) Plant Patent Application Publication
(10) Pub. No.: US 2026/0130303 P1
(43) Pub. Date: May 7, 2026

(54) ORANGE PLANT NAMED 'ORANGE 14'

(50) ;

(71) Applicant: Florida Foundation Seed Producers, Inc., Marianna, FL (US)

(72) Inventor: Jude GROSSER, Winter Haven, FL (US)

(21) Appl. No.: 19/381,498

(22) Filed: Nov. 6, 2025

Related U.S. Application Data
(60) Provisional application No. 63/717,582, filed on Nov. 7, 2024.

(51) Int. Cl. A01H 6/78; A01H 5/02; A01H 5/08

(52) U.S. Cl. CPC A01H 6/785; A01H 5/02; A01H 5/08
USPC PLT/202

(57) ABSTRACT

A new and distinct cultivar of orange plant named ‘Orange 14’, an allotetraploid rootstock with improved disease resistance for use in HLB-endemic areas. This cultivar produces adequate nucellar seed making it amenable to standard nursery propagation practices for uniform liner production. Ungrafted seed trees are especially tolerant of HLB.

### CROSS-REFERENCE TO RELATED APPLICATIONS

This application claims the benefit of U.S. Provisional Patent Application No. 63/717,582, filed Nov. 7, 2024, entitled ORANGE PLANT NAMED ‘ORANGE 14’, which is incorporated by reference herein in its entirety.

### ACKNOWLEDGEMENT OF FEDERAL RESEARCH SUPPORT

N/A.

Genus and species:

Citrus sp. This new cultivar is a complex citrus hybrid containing Citrus grandis, Citrus paradisi, Citrus reticulata, and Citrus trifoliata.

Cultivar denomination:

‘Orange 14’.

### BACKGROUND OF THE NEW CULTIVAR

The present invention relates to a new and distinct cultivar of orange, botanically known as Citrus sp., and hereinafter referred to by the name ‘Orange 14’. This Citrus sp. is a member of Rutaceae.

The new cultivar of orange ‘Orange 14’ is an allotetraploid zygotic hybrid (“tetrazyg”) derived from a conventional cross of two somatic hybrids previously produced by protoplast fusion. The somatic hybrid seed parent is ‘Nova’ (not patented) mandarin hybrid + ‘Hirado Buntan’ (not patented) pummelo (zygotic seedling) and the somatic hybrid pollen parent is ‘Cleopatra’ (not patented) mandarin+‘Argentine’ (not patented) trifoliate orange. The new cultivar ‘Orange 14’ is a sibling of ‘UFR-1’ (U.S. Plant Pat. No. 27,277), ‘UFR-2’ (U.S. Plant Pat. No. 27,742), ‘UFR-4’ (U.S. Plant Pat. No. 27,745), and ‘Orange 16’ (not patented).

### SUMMARY OF THE INVENTION

The new sweet orange cultivar ‘Orange 14’ has not been observed under all possible environmental conditions. The phenotype of the new cultivar may vary with variations in environment and cultural practices such as temperature, light intensity, fertilization, irrigation, and application of plant growth regulators without any change in genotype.

‘Orange 14’ rootstock was selected on the basis of its positive reaction to HLB disease (huanglongbing or citrus greening disease) in multiple field trials. Trees grown on this rootstock show an ability to grow off into productive trees in HLB-endemic Florida with no individual protective covers (IPCs) and, in one trial (St. Cloud, Florida), no psyllid control. In a commercial Citrus Research and Field Trial (CRAFT), 4-year-old trees with ‘OLL-8’ sweet orange scions are performing well in an area with heavy Diaprepes/Phytophthora complex pressure. In one trial, the new cultivar ‘Orange 14’ out-performed other top commercial rootstocks in a wet flatwoods growing block, including ‘US-942’ (not patented), ‘US-812’ (not patented), ‘x639’ (not patented), ‘UFR-4’ (U.S. Plant Pat. No. 27,745), and ‘UFR-5’ (U.S. Plant Pat. No. 27,298). In another trial, the new cultivar ‘Orange 14’ performed very well under heavy HLB-pressure that included many rootstocks, planted near Babson Park, Florida. Seven-year-old trees with ‘OLL-10’ (not patented) sweet orange as the scion are also performing well in in other areas of Florida. Although trees grafted to ‘Orange 14’ rootstock grow off quickly, trees grow to medium size. Some roguing is required in the nursery, but this selection produces adequate nucellar seed making it amenable to standard nursery propagation practices for uniform liner production. Ungrafted seed trees are especially tolerant of HLB.

There are limited replicated field trial data regarding the performance of ‘Orange 14’ rootstock. However, trees grafted with ‘Valencia’ (not patented) and planted in 2008 in St. Cloud, Florida, grown with no psyllid control, are still productive. Seven-year-old trees with ‘Orange 14’ rootstock and ‘OLL-10’ scion planted near Lake Alfred, Florida, are also healthy and productive. One 4-tree plot was observed in Dundee, Florida, which did not perform well, but no specific reason for this was determined. Overall, ‘Orange 14’ rootstock appears to be at least as strong as any available commercial rootstocks in HLB-endemic Florida.

The new cultivar ‘Orange 14’ can be compared to the commercial cultivar ‘UFR-4’ The new cultivar ‘Orange 14’ has similar performance, but is stronger in areas impacted by the sugar cane root weevil (Diaprepes abbreviates), an insect pest that damages citrus roots.

### DESCRIPTION OF THE FIGURES

This new sweet orange cultivar ‘Orange 14’ is illustrated by the accompanying photographs which show the tree's from, foliage and fruit. The colors shown are as true as can be reasonably obtained by conventional photographic procedures. The photographs are of a tree approximately 6 years old.

FIG. 1 shows HLB-tolerant seed tree of the new cultivar ‘Orange 14’ planted in Lake Alfred, Florida;

FIG. 2 shows a seed tree of the new cultivar ‘Orange 14’ planted in Lake Alfred, Florida;

FIG. 3 shows seed fruit cluster in a seed tree of the new cultivar ‘Orange 14’;

FIG. 4 shows new leaf flush in a seed tree of the new cultivar ‘Orange 14’;

FIG. 5 shows leaf morphology of the new cultivar ‘Orange 14’;

FIG. 6 shows flower morphology of the new cultivar ‘Orange 14’;

FIG. 7 shows flower and fruit setting of the new cultivar ‘Orange 14’;

FIG. 8 shows flower size of the new cultivar ‘Orange 14’, with a ruler shown for size reference;

FIG. 9 shows a flower of the new cultivar ‘Orange 14’, showing dehiscing pollen with a ruler shown for size reference;

FIG. 10 shows a cut seed fruit of the new cultivar ‘Orange 14’;

FIG. 11 shows a cut seed fruit and extracted polyembryonic seed of the new cultivar ‘Orange 14’;

FIG. 12 shows typical polyembryonic seed of the new cultivar ‘Orange 14’;

FIG. 13 shows ‘Valencia’ grown on ‘Orange 14’ rootstock in St. Cloud, Florida, planted between two ‘Valencia’/rough lemon trees;

FIG. 14 shows ‘OLL-10’ sweet orange grown on ‘Orange 14’ rootstock near Lake Alfred, Florida;

FIG. 15 shows ‘OLL-8’ sweet orange grown on ‘Orange 14’ rootstock, 3.5-year-old planted commercial grove with heavy Diaprepes/Phytophthora pressure; and

FIG. 16 is a graph showing mean yield and cumulative yield (boxes/tree) by rootstock across all scions (8 OLL clones), sorted by cumulative yield. Trees were planted in summer of 2013 in St. Cloud, Florida, and grown with no psyllid control.

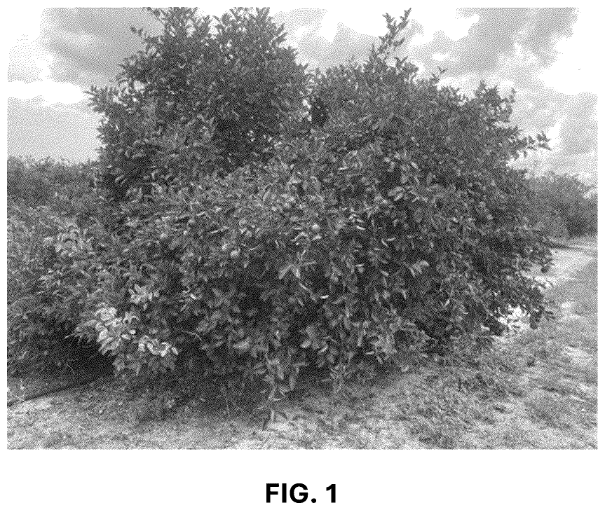

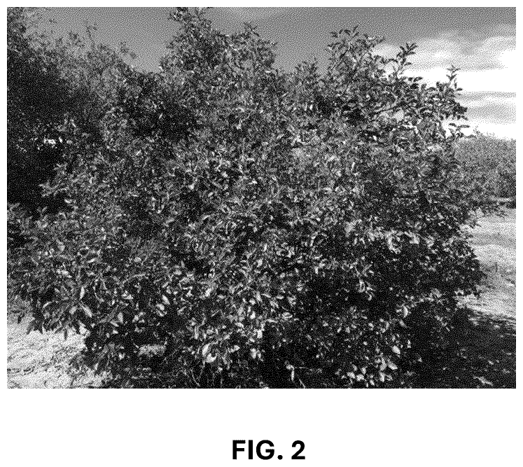

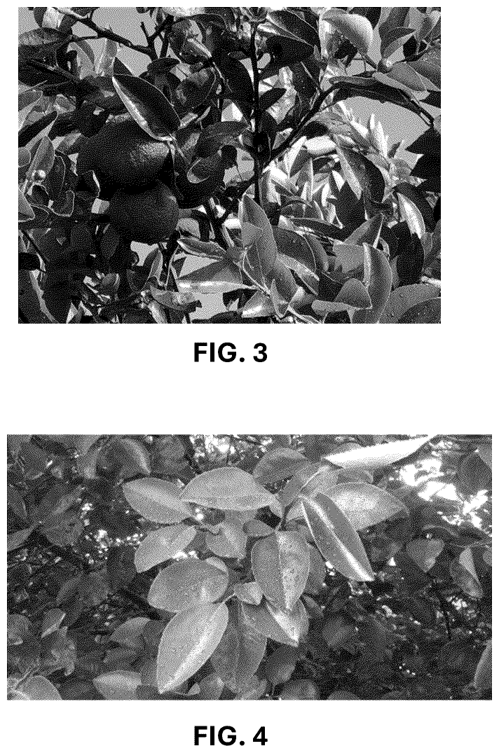

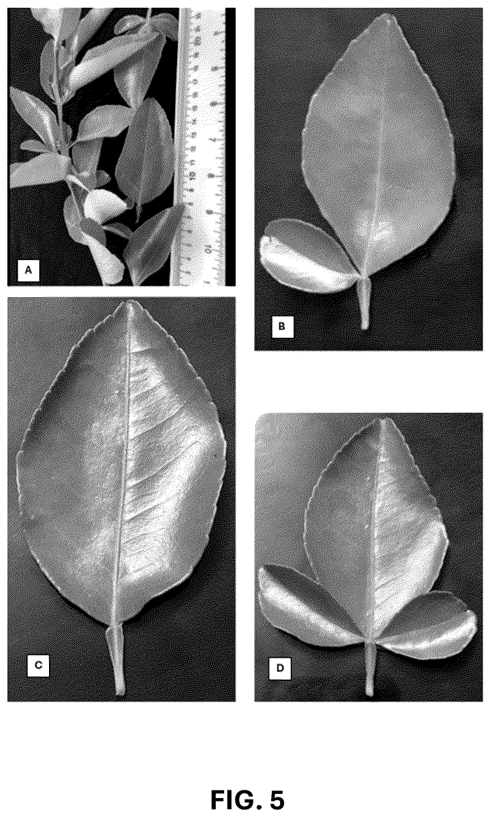

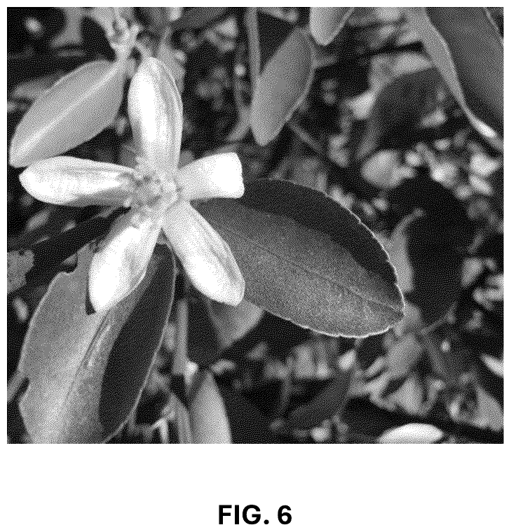

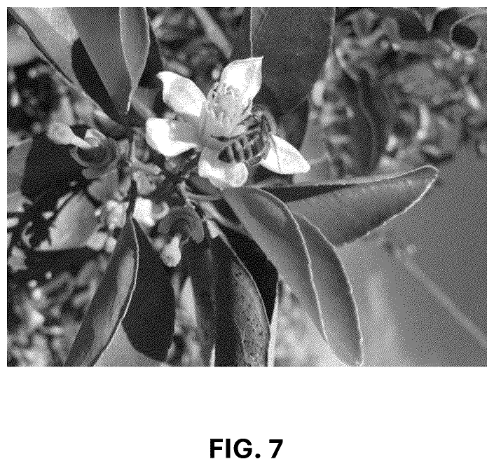

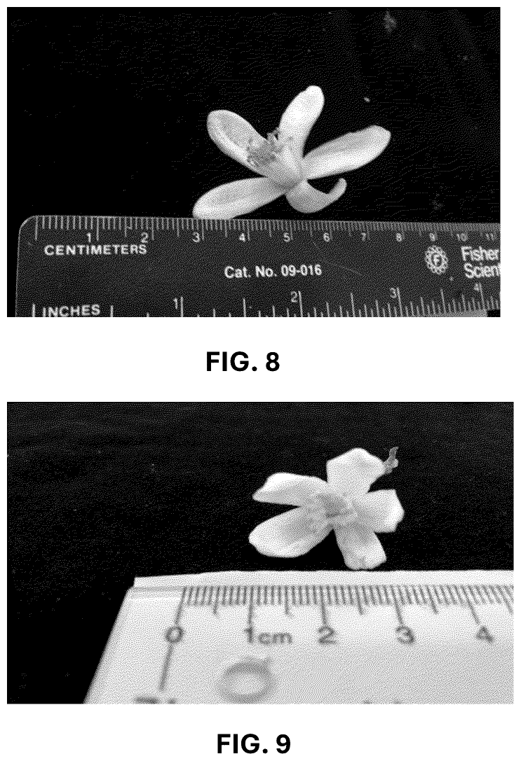

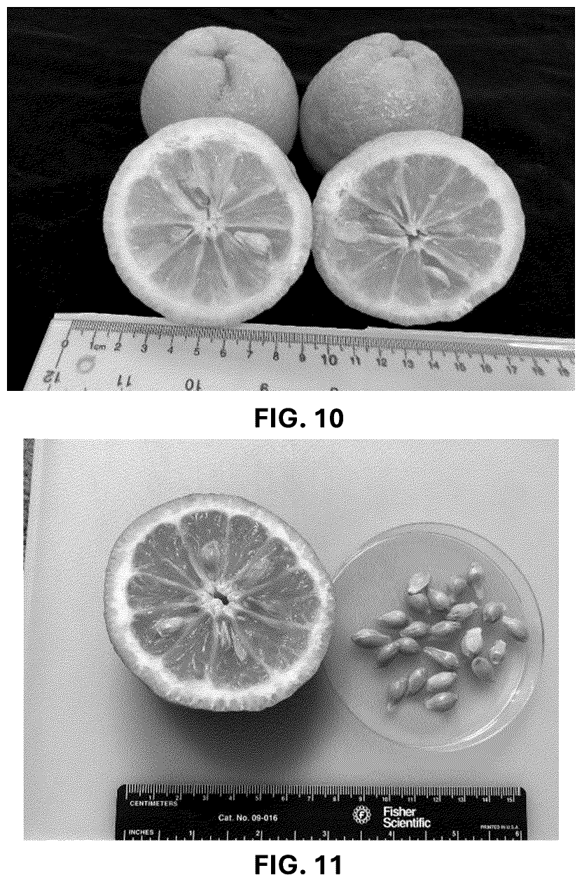

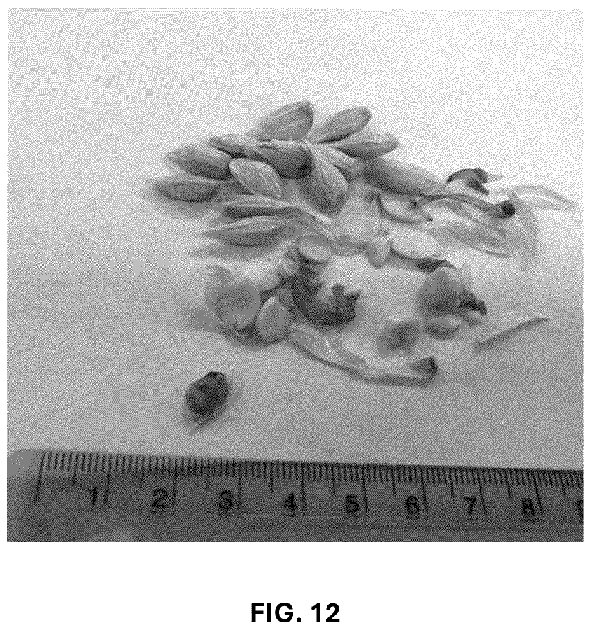

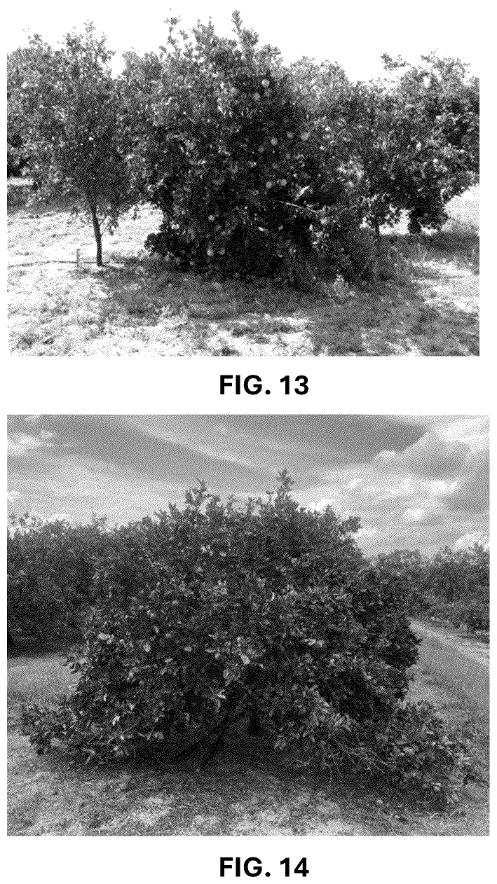

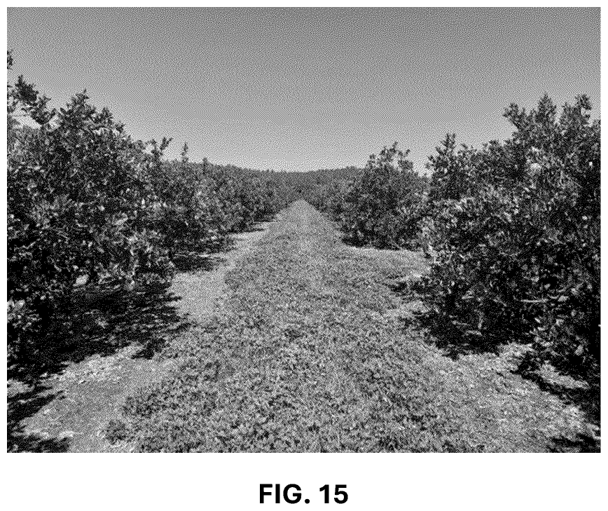

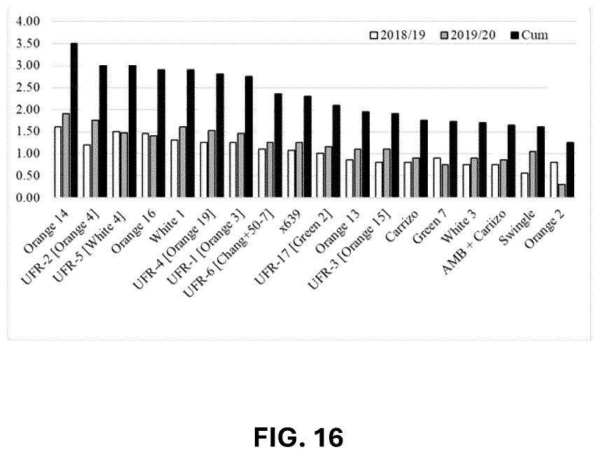

### DETAILED BOTANICAL DESCRIPTION OF THE CULTIVAR

### Botanical Description

Botanical classification:
   Family.—Rutaceae.
   Botanical name.—Citrus sp. This new cultivar is a complex citrus hybrid containing Citrus grandis, Citrus paradisi, Citrus reticulata, and Citrus trifoliata.
   Common name.—Tetrazyg rootstock.
   Cultivar.—‘Orange 14’.
Parentage: The somatic hybrid seed parent is ‘Nova’ mandarin hybrid+‘Hirado Buntan’ pummelo (zygotic seedling) and the somatic hybrid pollen parent is ‘Cleopatra’ mandarin+‘Argentine’ trifoliate orange.
Tree:
   Ploidy.—Tetraploid.
   Size.—Medium, moderate.
   Width across the row.—3.5 meters on average.
   Width down row.—3.5 to 4 meters on average.
   Tree Height.—3.5 meters on average
   Tree spread.—Up to 4.00 meter.
   Vigor.—Moderate to vigorous
   Density.—Canopies are quite dense.
   Tree shape (form).—The shape of the tree is obloid with lateral branches.
   Tree diameter.—4 meters on average.
   Growth habit (current season).—Mostly lateral with some upright growth.
Trunk:
   Trunk diameter.—Approximately 10.5 cm in diameter at 30 cm above from ground in a 6-year-old tree.
   Trunk texture.—Smooth.
   Trunk bark color.—RHS N199A (Grey-Brown).
Branches:
   Branch length.—2.5 to 3 meters on average
   Crotch angle.—Acute (less than 90 degrees).
   Branch texture.—Relatively smooth with some thorns.
   Branch color.—RHS 137A (Green Group) with some RHS 137B (Green Group).
   Spine size.—10-20 mm on average.
   Spine color.—RHS 137A (Green Group) with some RHS N199B (Greyed-Brown) based on the age of the branch
Leaves:
Size (lamina average):
   Length.—90 mm on average.
   Width.—50 mm on average
   L/W ratio.—1.8.
   Thickness.—Medium (approximately 0.23 mm).
   Type.—The tree has three types of leaves.—unifoliate, bifoliate and trifoliate.
   Shape.—Elliptical.
   Apex.—Retuse.
   Base.—Acute.
   Margin.—Dentate.
Surface:
   Upper surface.—Smooth.
   Lower surface.—Smooth with the medium veins that are pinnately netted.
Color:
   Upper surface (adaxial).—RHS 137A (Green Group).
   Lower surface (abaxial).—RHS 137B (Green Group).
Petiole:
   Shape and attachment.—Brevipetiolate (shorter than leaf lamina); junction between petiole and lamina is articulate.
   Width.—Narrow.
   Shape (petiole wing).—Obovate.
   Length.—18 mm to 22 mm.
   Width.—3 mm to 3.2 mm.
   Color.—RHS 137A (Green).
Flowers:
   Type.—Hermaphrodite.
   Flowers bearing.—Single or cluster. Branches have flowers grown in clusters. Each cluster consists of 2-5 flowers. Individual flowers grow from leaf terminals and leaf axillaries.
   Flower diameter.—Fully open flower with average diameter of 25 mm to 35 mm.
   Flower depth.—Typical flower with average depth of 20 mm.
Flower blooming period:
   First bloom observed.—Late February 2024.
   Full bloom observed.—Mid-March 2024.
Flower bud size:
   Length.—Initial visible flower bud is approximately 2 mm in length; mature flower bud is approximately 20 mm in length.
   Diameter.—Initial visible flower bud is approximately 2 mm in diameter; mature flower bud is approximately 5.5 mm in diameter
   Shape.—Initial visible flower bud has a round ball shape; mature flower bud has an elongated olive shape.
   Color.—RHS 144C (Yellow-Green) for initial visible flower bud; RHS NN 155A (White) for mature flower bud.
Petals:
   Number.—5 per flower.
   Shape.—Flat spatula shaped.
   Length.—22 mm on average.
   Width.—6 mm on average.
   Apex shape.—Smooth, acute shaped.
   Base shape.—Even obtuse.
   Color.—Upper surface is RHS NN 155A (White); lower surface is RHS NN 155C (White).
   Margin.—Smooth.
Sepal:
   Number.—3-5 per flower.
   Shape.—Delta shaped with acute angle at apex.
   Length.—3 mm on average.
   Width.—3.5 mm on average.
   Apex shape.—Triangle shaped.
   Margin.—Smooth.
   Color.—RHS 1A (Green-Yellow).
   Fragrance.—Moderately fragrant.
Pedicel:
   Length.—7 mm to 8 mm.
   Diameter.—2 mm on average.
   Color.—RHS 1B (Green-Yellow).
Stamen:
   Number.—22 per flower on average.
   Length.—13 mm on average.
Anther:
   Length.—2.5 mm on average.
   Width.—1.5 mm on average.
   Color.—RHS 10A (Yellow).
   Pollen amount.—Abundant.
   Pollen color (general).—Bright yellow, RHS 17A (Yellow).
Pistil:
   Number.—1.
   Length.—12.5 mm on average.
   Color.—RHS 153B (Yellow-Green).
   Style length.—9.5 mm on average.
   Style diameter.—1 mm on average.
   Style color.—RHS 144A (Yellow-Green).
   Ovary shape.—Oval.
   Ovary diameter.—2.5 mm on average.
   Ovary color.—RHS N 144D (Yellow-Green).
Fruit:
   Fruit parthenocarpy.—Yes.
Size: Uniform.
   Tall.—75 mm to 82 mm on average.
   Diameter.—75 mm on average.
   Average weight (per individual fruit).—194.73 grams.
   Shape.—Round.
   Shape (cross-section).—Round.
   Apex.—Truncated.
   Base.—No neck, slightly rough peel surface.
Harvest:
   First pick.—Around September.
   Last pick.—Around December.
Fruit stem (short stem connecting the fruit):
   Length.—8.5 mm on average.
   Diameter.—3.5 mm on average.
   Color.—RHS 198D (Greyed-Green).
Rind:
   Adherence.—Adherence between albedo (mesocarp) and flesh (endocarp) is medium.
   Thickness.—4 mm on average.
   Texture.—Firmed.
Color:
   Flavedo (epicarp).—RHS 163B (Greyed-Orange).
   Albedo (mesocarp).—RHS NN 150B (White).
   Style end.—Closed.
   Rind oil cell density.—Approximately 180 cells/cm².
Flesh:
   Number of segments.—10-12 segments per fruit.
   Segment walls.—Medium.
   Juice.—Abundant.
   Color.—RHS 163C (Greyed-Orange) with some RHS 10A (Yellow Group).
   Texture.—Soft.
Vesicles: Medium thickness.
   Length.—13 mm to 15 mm on average.
   Diameter.—2.5 mm to 3 mm on average.
Seeds:
   Type.—Polyembryonic.
   Number.—18 to 22 per fruit.
   Shape.—Seed shapes are uniform, elongated oval shape.
Size:
   Length.—15 mm to 17 mm.
   Width.—9 mm to 10 mm.
   Seed coat color.—RHS 158B (Yellow-White).
   Outer Surface.—RHS 161B (Greyed-Yellow).
   Inner surface.—RHS 165C (Greyed-Orange).
   Cotyledon color.—RHS 157D (Green-White).

### Data:

TABLE 1
St. Cloud Demonstration Planting:
2018/2019 and 2019/2020 PS (lbs. solids)/tree and 2-year
cumulative yield by rootstock for ‘OLL-8’ sweet orange.

| | PS/Tree | | |
|---|---|---|---|
| Rootstock | 2018/2019 | 2019/2020 | Cumulative |
| ‘Orange 14’ | 13.1 | 17.0 | 30.1 |
| ‘UFR-5’ (White 4) | 17.1 | 12.9 | 30.1 |
| ‘UFR-1’ (Orange 3) | 13.2 | 12.3 | 25.6 |
| ‘UFR-2’ (Orange 4) | 11.4 | 13.9 | 25.4 |
| ‘Orange 16’ | 13.3 | 11.2 | 24.5 |
| ‘White 1’ | 11.6 | 11.1 | 22.7 |
| ‘UFR-4’ (Orange 19) | 8.6 | 11.6 | 20.1 |
| ‘UFR-6’ | 9.6 | 8.3 | 17.9 |
| (‘Chang’ + ‘50-7’) | | | |
| ‘x639’ | 8.7 | 7.9 | 16.6 |
| ‘Carrizo’ | 9.3 | 6.2 | 15.5 |
| ‘Orange 2’ | 8.5 | 6.8 | 15.3 |
| ‘Swingle’ | 6.9 | 8.2 | 15.1 |
| ‘Orange 13’ | 6.7 | 6.0 | 12.6 |
| ‘White 3’ | 5.1 | 7.5 | 12.6 |
| ‘UFR-3’ (Orange 15) | 5.9 | 6.6 | 12.4 |
| ‘UFR-17’ (Green 2) | 6.5 | 5.6 | 12.0 |
| ‘AMB’ + ‘Carrizo’ | 3.2 | 3.5 | 6.7 |
| ‘Green 7’ | 3.5 | N/A | 3.5 |

Trees were planted in summer of 2013 in St. Cloud, Florida, and grown with no psyllid control.

1. A new and distinct orange plant named ‘Orange 14’ as illustrated and described herein.
